(19) United States
(12) Patent Application Publication
(10) Pub. No.: US 2024/0271147 A1
(43) Pub. Date: Aug. 15, 2024

(54) TOBAMOVIRUS PSEUDOVIRIONS FOR STABILISING SINGLE STRANDED RNA

(71) Applicant: University of Cape Town, Cape Town (ZA)

(72) Inventors: Ann Elizabeth MEYERS, Cape Town (ZA); Edward Peter RYBICKI, Cape Town (ZA); Inga Isabel Hitzeroth, Cape Town (ZA); Susan Jennifer Dennis, Cape Town (ZA)

(73) Assignee: University of Cape Town, Cape Town (ZA)

(21) Appl. No.: 18/568,456

(22) PCT Filed: Jun. 10, 2022
(86) PCT No.: PCT/IB2022/055404

§ 371 Date: Dec. 8, 2023

(30) Foreign Application Priority Data
Jun. 11, 2021 (GB) 2108392.8

(51) Int. Cl. C12N 15/82; C07K 14/005

(52) U.S. Cl. CPC C12N 15/8201; C07K 14/005; C12N 2770/00022; C12N 2770/00023; C12N 2770/00034; C12N 2770/00042

(57) ABSTRACT

Provided herein is a method for stabilising a single stranded RNA (ssRNA) by encapsidation of the ssRNA with a tobamovirus coat protein to obtain a pseudovirion (PsV), the method comprising expressing a tobamovirus coat protein and the ssRNA comprising a tobamovirus encapsidation origin (OriA), wherein the expressed tobamovirus coat protein interacts with the OriA sequence on the ssRNA to initiate encapsidation of the ssRNA by the tobamovirus coat protein, thereby forming a pseudovirion. The PsVs produced according to the method can be used as a diagnostic control composition, where the ssRNA is a sequence detected by a molecular diagnostic assay. The pseudovirions may also be used as a vaccine to elicit an immune response in a subject, and in pharmaceutical compositions to be administered to a subject.

### CROSS-REFERENCE TO RELATED PATENT APPLICATIONS

This application is a U.S. National Stage Application under 35 U.S.C. § 371 of International Patent Application No. PCT/IB2022/055404 filed Jun. 10, 2022, which claims the benefit of priority of Great Britain Application No. 2108392.8 filed Jun. 11, 2021, both of which are incorporated by reference in their entireties. The international application was published on Dec. 15, 2022, and received publication no.

WO/2022/259215.

### SEQUENCE LISTING

The instant application contains a Sequence Listing which has been submitted electronically in ASCII format and is hereby incorporated by reference in its entirety. Said ASCII copy, created on Dec. 8, 2023, is named “CMP-Sequence Listing-12380-012283-USO” and is 15,460 bytes in size.

### BACKGROUND OF THE INVENTION

The present invention relates to methods of stabilising a single stranded RNA (ssRNA) by encapsidation of the ssRNA with a tobamovirus coat protein (CP) to obtain pseudovirions (PsVs) which enclose an artificial pseudogenome comprising the stabilised ssRNA. The present invention further relates to PsVs produced according to the method, to a diagnostic control composition comprising the PsVs and to vaccines and pharmaceutical compositions comprising the PsVs.

Accuracy of testing for viral infections is critical in managing and treating viral infectious diseases. Real-time reverse transcription polymerase chain reaction (RT-PCR) assays are the tool of choice for detecting active viral shedding of RNA viruses. RT-PCR assays combine reverse transcription of viral RNA into DNA followed by amplification of specific targets by polymerase chain reaction (PCR). PCR is extremely sensitive and requires only trace amounts of the template DNA or RNA for amplification and identification. When used for pathogen detection, RT-PCR assays require the use of appropriate controls. Such controls aid in the interpretation of results by identifying contamination during processing, inhibition of the reverse transcription and amplification reactions, identifying whether the template DNA or RNA extraction was successful, and testing of instrument or assay suitability.

The recently emergent severe acute respiratory syndrome coronavirus 2 (SARS-CoV-2) is the virus that causes the infectious disease COVID-19, which has resulted in a global pandemic. Common symptoms of COVID-19 include fever, cough and shortness of breath. Other symptoms may include fatigue, muscle pain, diarrhea, sore throat, loss of smell and abdominal pain. The time from exposure to the onset of symptoms is typically five days but may range from two to fourteen days. Primarily the virus is spread between people when they come into close contact, via small droplets produced by coughing, sneezing, or talking. While these droplets are produced when breathing out, and remain suspended for some time, they eventually fall to the ground or onto surfaces rather than remain in the air over long distances. People may also become infected by touching a contaminated surface and then touching their eyes, nose, or mouth. A major challenge in containing the spread of the virus is identifying asymptomatic infections which are reported to be major drivers of the pandemic and identifying potential animal reservoirs to prevent future outbreaks.

With the COVID-19 pandemic, as well as many other viral infections, it has been demonstrated that smart, aggressive public health interventions can reduce the rate of new infections. For example, in the recent outbreak countries such as China and South Korea have been able to contain the spread of new infections through rapidly deploying testing for COVID-19 infection and disease. When these diagnostics were coupled with effective public health interventions such as isolation and quarantine, a significant flattening of the curve was achieved.

RT-PCR tests have the capacity for detecting virus early in an infection, in individuals who are asymptomatic, and allows for reduction in spread of new infections. As such the demand for RT-PCR tests has increased exponentially all over the world. However, as these become available the need for both positive and negative controls also becomes an urgent priority. Proficiency testing controls allow for an assessment of whether a test is fit for purpose and provides valuable information on the ability of laboratories to carry out testing in a quality assured manner. These controls usually encompass both positive and internal controls. Positive controls prevent false negative results from occurring if the reverse transcription and/or PCR reactions are not functioning properly. Internal control systems may include the addition of exogenous RNA to a sample prior to extraction. In this way, amplification of the control indicates that the extraction was successful and that the RT-PCR method was successful.

However, RNA is more chemically labile than DNA. Specifically, the sugar-phosphate backbone of RNA contains ribose instead of DNA, which contains deoxyribose. Ribose has a hydroxyl group attached to the pentose ring in the 2′ position, whereas deoxyribose does not. The hydroxyl groups in the ribose backbone make RNA more chemically labile than DNA by lowering the activation energy of hydrolysis. Thus, RNA is highly reactive, unstable and easily degradable. There is thus a need to provide stabilised RNA for use as controls for RT-PCR tests.

In addition, mRNA vaccines have been cited as novel and exciting prospects in vaccinology for some time. However, concerns over mRNA instability, high innate immunogenicity and inefficient in vivo delivery have slowed their delivery into humans. RNA vaccines were the surprising early leaders of efforts to make vaccines against SARS-CoV-2, with two candidates (Moderna and BioNTech) made in vitro using modified nucleotides showing good efficacy, albeit with considerable reactogenicity. A major drawback for the wider application of mRNA vaccines, however, is their complexity of manufacture and significant bottlenecks in supply chains for the reagents required.

The inventors have developed tobamovirus pseudovirion (PsV) nanoparticles comprising tobamovirus coat proteins (CP) which enclose an artificial pseudogenome comprising target sequences for PCR- or LAMP-based amplification techniques for the detection of single stranded RNA in samples, as well as an indicator gene. The TMV PsVs of the invention may be used as positive and internal diagnostic controls in PCR- or LAMP-based amplification assays. Alternatively, the TMV PsVs may find application as viral vaccines.

### SUMMARY OF THE INVENTION

According to the present invention there is provided for a method for stabilising a single stranded RNA (ssRNA) by recombinant encapsidation of the ssRNA with a tobamovirus coat protein to obtain a tobamovirus pseudovirion (PsV), wherein the method comprises or consists of expressing a tobamovirus coat protein (CP), such as a Tobacco mosaic virus (TMV) CP, and a single stranded RNA of interest comprising a tobamovirus encapsidation origin (OriA), wherein the expressed CP interacts with the OriA sequence on the ssRNA to initiate encapsidation of the ssRNA by the CP, thereby forming a pseudovirion. The present invention further relates to diagnostic control compositions comprising the PsVs produced according to the method, where the ssRNA is a stabilised ssRNA comprising a sequence detected by a molecular diagnostic assay. The pseudovirions may also be used as stabilised mRNA vaccines to elicit an immune response in a subject, and in pharmaceutical compositions to be administered to a subject.

According to a first aspect of the present invention there is provided for a method for stabilising a single stranded RNA (ssRNA) by encapsidation of the ssRNA with a tobamovirus coat protein to obtain a pseudovirion (PsV), the method comprising the steps of: providing a first nucleic acid encoding a tobamovirus coat protein, such as a Tobacco mosaic virus (TMV) coat protein and a second nucleic acid encoding a single stranded RNA (ssRNA) comprising a tobamovirus encapsidation origin (OriA), such as a Tobacco mosaic virus (TMV) OriA, wherein the first and second nucleic acids are contained on at least one expression vector; expressing the coat protein; and co-expressing the ssRNA, wherein the expressed coat protein interacts with the OriA sequence on the ssRNA to initiate encapsidation of the ssRNA by the coat protein, thereby forming a pseudovirion.

In a first embodiment of the method for stabilising a ssRNA, the first and second nucleic acids are operably linked to regulatory sequences that allow for expression of the coat protein and the ssRNA, respectively.

According to a third embodiment of the method for stabilising a ssRNA, the ssRNA may be one or more ssRNA selected from the group consisting of: a mRNA encoding a reporter gene, a ssRNA target of a molecular diagnostic assay for the detection of a ssRNA virus, a mRNA target of a diagnostic assay for the detection of a disease or disorder, mRNA encoding a therapeutic gene, mRNA encoding an antigenic polypeptide, mRNA encoding a hormone, mRNA encoding an antibody and mRNA encoding an enzyme. It will be appreciated by those of skill in the art that many reporter genes are known in the art. In one non-limiting embodiment, the reporter gene may be selected from an enhanced green fluorescent protein gene, luciferase gene, a secreted alkaline phosphatase gene, a gene encoding a fluorescent protein or a horseradish peroxidase gene. The method of the present invention may find application in the field of molecular diagnostic assays, wherein the ssRNA is a ssRNA target of a molecular diagnostic assay for the detection of a ssRNA virus, including a ssRNA virus sequence detected by the molecular diagnostic assay, such as one or more sequence of a virus selected from the group consisting of an influenza virus, a parainfluenza virus, a respiratory syncytial virus, a measles virus, a coronavirus, a rhinovirus, an adenovirus, HCV, HIV, and Ebola virus. In one embodiment, the ssRNA virus sequence may be a SARS-CoV-2 sequence, such as a sequence of the SARS-CoV-2 RdRp, N, E and/or S genes, and including a SARS-CoV-2 sequence of SEQ ID NO:1. Alternatively, the molecular diagnostic assay may be a molecular diagnostic assay for detecting a disease or disorder, wherein the ssRNA is a mRNA target of a diagnostic assay for the detection of the disease or disorder.

In a fourth embodiment of the method for stabilising a ssRNA, the tobamovirus coat protein and the ssRNA may be expressed in a plant cell and the method may further comprise recovering the PsVs from the plant cell. In a preferred embodiment of the method of the invention, the plant cell may be a Nicotiana benthamiana plant cell.

According to a second aspect of the present invention there is provided for a pseudovirion produced according to the method for stabilising a ssRNA by recombinant encapsidation of the ssRNA with a tobamovirus coat protein to obtain a pseudovirion, wherein the pseudovirion comprises or consists of a tobamovirus coat protein capsid which encapsidates the ssRNA of interest comprising the OriA.

In a third aspect of the present invention there is provided for a diagnostic control composition comprising a tobamovirus pseudovirion, in particular a tobamovirus pseudovirion produced according to the method for stabilising a ssRNA by recombinant encapsidation of the ssRNA with a tobamovirus coat protein to obtain a pseudovirion, wherein the pseudovirion comprises or consists of a tobamovirus coat protein capsid which encapsidates the ssRNA comprising a tobamovirus encapsidation origin (OriA), wherein the ssRNA is a sequence detected by a molecular diagnostic assay and wherein the diagnostic control composition is a positive control for the molecular diagnostic assay. In some embodiments, the tobamovirus coat protein is a Tobacco mosaic virus coat protein and the OriA is a Tobacco mosaic virus OriA.

In a first embodiment of the diagnostic control composition, the ssRNA of interest may be one or more ssRNA selected from the group consisting of a mRNA encoding a reporter gene, a ssRNA target of a molecular diagnostic assay for the detection of a ssRNA virus, and a mRNA target of a diagnostic assay for the detection of a disease or disorder. It will be appreciated by those of skill in the art that numerous reporter genes are known. In one non-limiting embodiment, the reporter gene may be selected from an enhanced green fluorescent protein gene, luciferase gene, a secreted alkaline phosphatase gene, a gene encoding a fluorescent protein or a horseradish peroxidase gene.

In a second embodiment of the diagnostic control composition of the invention, the ssRNA target of the molecular diagnostic assay for the detection of a ssRNA virus may be an ssRNA virus sequence detected by the molecular diagnostic assay. In one non-limiting example, the ssRNA virus sequence may be one or more sequence of a virus selected from the group consisting of an influenza virus, a parainfluenza virus, a respiratory syncytial virus, a measles virus, a coronavirus, a rhinovirus, an adenovirus, HCV, HIV, and Ebola virus. For example, the ssRNA virus sequence may be a SARS-CoV-2 sequence, such as a sequence of the SARS-CoV-2 RdRp, N, E and/or S genes, and including a SARS-CoV-2 sequence of SEQ ID NO:1.

According to a fourth aspect of the present invention there is provided for a pharmaceutical composition comprising a pseudovirion produced according to the method for stabilising a ssRNA by recombinant encapsidation of the ssRNA with a tobamovirus coat protein, such as a Tobacco mosaic virus coat protein, to obtain a pseudovirion, wherein the pseudovirion comprises or consists of a tobamovirus coat protein capsid which encapsidates the ssRNA of interest comprising the OriA, together with a pharmaceutically acceptable carrier or adjuvant.

In a further aspect of the present invention there is provided for a pseudovirion produced according to the method for stabilising a ssRNA by recombinant encapsidation of the ssRNA with a tobamovirus coat protein to obtain a pseudovirion, wherein the pseudovirion comprises or consists of a tobamovirus coat protein capsid which encapsidates the ssRNA of interest comprising the OriA, for use in a method of eliciting an immune response in a subject, wherein the method of eliciting an immune response in a subject comprises or consists of administering the pseudovirion to the subject. In some embodiments the tobamovirus coat protein is a Tobacco mosaic virus coat protein and the OriA is a Tobacco mosaic virus OriA.

The present invention further provides for kits comprising the pseudovirion produced according to the method for stabilising a ssRNA by recombinant encapsidation of the ssRNA with a tobamovirus coat protein, such as a Tobacco mosaic virus coat protein, to obtain a pseudovirion, as well as for kits comprising the diagnostic control compositions containing said pseudovirions.

### BRIEF DESCRIPTION OF THE FIGURES

Non-limiting embodiments of the invention will now be described by way of example only and with reference to the following figures:

FIG. 1: Design and synthesis of the SARS-CoV2 and RNAse P DNA concatenated sequence (SEQ ID NO:1).

FIG. 2: Construction of Agrobacterium recombinant strains: A—Restriction enzyme digestion analysis to confirm successful cloning of EGFP_SARS-CoV2_conc_OriA and TMV CP into the pRic4 plant transient expression vector. B—Colony PCR to demonstrate successful electroporation of pRIC4_TMV_CP and pRIC4_EGFP_SARS-CoV2_conc_OriA into Agrobacterium strain GV3101::pMP90RK.

FIG. 3: EGFP expression in co-infiltrated leaf tissue: A—Plant-expressed EGFP (±26 kD) was precipitated from infiltrated crude leaf extract by sequential addition of 20% (lane 2), 40% (lane 3), 60% (lane 4) and 80% (NH₄)₂SO₄ and analysed by western blot using a commercial anti EGFP serum (1:10 000) raised in mice. Crude extract prior to (NH₄)₂SO₄ precipitation was used to demonstrate EGFP expression in the plants (lane 5), while crude extract from plants infiltrated with an Agrobacterium construct containing the empty pRic4 vector is shown in lane 6. Molecular weight marker sizes are shown on the left. B—pRIC4_EGFP_SARS_CoV2_conc_OriA/GV3101 and pRIC4_TMV_CP/GV3101 co-infiltrated leaf tissue viewed under long wave-length UV light at 3 days post infiltration.

FIG. 4: Purification of TMV SARS-CoV2 PsVs: PEG precipitates of crude extracts from leaves co-infiltrated with the pRIC4_EGFP_SARS_CoV2_conc_OriA/GV3101 and pRIC4_TMV_CP/GV3101 Agrobacterium recombinants were analysed by SDS-PAGE and western blot using TMV antiserum (1:5000) as the primary antibody. Molecular weight markers are shown to the left and right of the gel and blot. A protein band of the appropriate 16 kD size of the TMV coat protein monomer was visualized in each case.

FIG. 5: Transmission Electron Micoroscopy (TEM) analysis of the PEG-precipitated preparations revealed the presence of rod-shaped TMV SARS-CoV2 PsVs varying in length from the expected size of ±100 nm to much longer rods, believed to be end-to-end arrangements of the TMV PsVs. Scale bars: 200 nm.

FIG. 6: PCR amplification of EGFP_SARS_CoV2_conc_OriA mRNA contained within the TMV SARS_CoV2 PsVs: Correctly sized 2177 bp (lane 4) and 231 bp (lane 3) fragments were transcribed and amplified using EGFP forward/OriA reverse primer set (SEQ ID NO:9 and SEQ ID NO:8) and OriA forward/OriA reverse primer set (SEQ ID NO:7 and SEQ ID NO:8), respectively. The 477 bp TMV CP gene (lane 2) was transcribed and amplified from TMV PsVs using the TMV CP forward/TMV CP reverse primer set (SEQ ID NO:5 and SEQ ID NO:6) as a positive control, while no amplification product resulted when the PsVs in the reverse transcription reaction were replaced with water (lane 1).

FIG. 7: General scheme of the RNA constructs used for obtaining the tobamovirus PsVs of the invention. TE=translational enhancer sequence. OriA=origin of assembly.

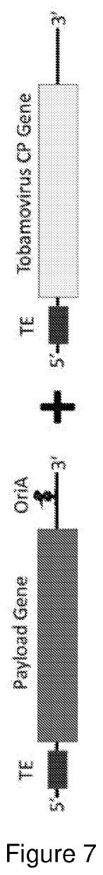

### SEQUENCE LISTING

The nucleic acid sequences listed in the accompanying sequence listing are shown using standard letter abbreviations for nucleotide bases. It will be understood by those of skill in the art that only one strand of each nucleic acid sequence is shown, but that the complementary strand is included by any reference to the displayed strand. In the accompanying sequence listing:

SEQ ID NO:1—nucleotide sequence of the artificial DNA sequence containing the SARS-CoV-2 target sequences

SEQ ID NO:2—nucleotide sequence of the reporter gene expressing EGFP

SEQ ID NO:3—nucleotide sequence derived from the TMV encapsidation origin (OriA)

SEQ ID NO:4—nucleotide sequence of the TMV coat protein (CP) gene

SEQ ID NO:5—nucleotide sequence of the TMV CP forward primer

SEQ ID NO:6—nucleotide sequence of the TMV CP reverse primer

SEQ ID NO:7—nucleotide sequence of the OriA forward primer

SEQ ID NO:8—nucleotide sequence of the OriA reverse primer

SEQ ID NO:9—nucleotide sequence of the EGFP forward primer

SEQ ID NO:10—nucleotide sequence of the 1455 bp sequence containing the Tomato Aspermy virus (TAV) 2b gene

SEQ ID NO:11—nucleotide sequence of the pRIC4 vector

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items.

The present invention, in its broadest sense, relates to the use of a plant virus coat protein to stabilize single-stranded RNA by encapsidation in a plant cell. More specifically, the inventors have successfully produced Tobacco Mosaic Virus pseudovirions that enclose a pseudogenome comprising target single stranded RNA sequences detected by nucleic acid-based detection systems, such as PCR- or LAMP-based amplification assays, and which function as a positive internal control for the assay. The tobamovirus PsVs provide a safe, stable and inexpensive reagent for use as a positive control in quantitating viral load, verifying assays, and aiding in cell culture work with live viruses. In addition, the purification of the tobamovirus PsVs is highly scalable and very cheap; the pseudovirions can be stored at 4° C. in liquid form indefinitely; and PCR tests on PsVs containing the TMV CP gene RNA only and stored for over 4 years at 4° C. showed that the RNA contained therein was still stable.

In one embodiment, the tobamovirus PsVs of the present invention may be used as both an internal and external control for single stranded RNA diagnostic assays. Specifically, the tobamovirus PsVs of the present invention may be used as an internal control for determining efficacy of RNA extraction and to detect the presence of inhibitors of PCR, however, the tobamovirus PsVs of the invention may also function a positive control for the binding of the specific primers or probes employed in the diagnostic assays. Further, according to the present invention, the clinical sample may either be spiked with the PsVs of the invention or the clinical sample and the PsVs may be tested side-by-side.

In the present invention, the inventors have designed and synthesised an artificial DNA sequence that contains numerous target sequences on the SARS-CoV-2 genome that are routinely used for RT-PCR or other infection diagnostic nucleic acid detection systems (SEQ ID NO:1). The sequence, flanked by a reporter gene expressing enhanced green fluorescent protein (EGFP) at the 5′ end (SEQ ID NO:2), and a sequence derived from the Tobacco Mosaic Virus (TMV) encapsidation origin (OriA) at the 3′ end (SEQ ID NO:3), has been cloned into a geminivirus-derived enhanced plant expression vector (pRIC4), which is a modified vector based on the pRIC3 vector previously described in Regnard et al (2010), which is incorporated herein in its entirety by reference. DNA was purified from recombinant Escherichia coli and this construct was then introduced into Agrobacterium tumefaciens bacteria and used for transient mRNA expression via agroinfiltration transformation techniques in the plant Nicotiana benthamiana. Plants were co-transformed with a pRIC4 Agrobacterium construct expressing the TMV coat protein (CP) gene (SEQ ID NO:4).

The mRNA was expressed at high levels from the pRic4 vector and shown to be encapsidated in planta by the TMV CP, which specifically interacts with and initiates assembly of virus-like particles at the OriA sequence present on each copy of the RNA. This assembly is highly efficient, very specific, and results in rodlike TMV PsV particles whose length is determined by the length of the RNA being encapsidated. Yield of such particles was high, and a simple purification strategy was employed. Generally, purified particle preparations and the encapsidated RNA are stable in suspension in a simple PBS or Tris buffer at 4° C. for years, with a half-life at 80° C. of 30 minutes. However, the inventors have shown that subjecting the particles to the sample buffers used for SARS-CoV-2 testing results in disruption of the CP-RNA association, permitting cDNA synthesis by reverse transcription of the encapsidated mRNA and allowing amplification of forward and reverse primer-defined DNA sequences.

The TMV SARS-CoV-2 PsV preparations described herein can be serially diluted in diagnostic kit patient swab sample buffers and amplified by the technique used in the test for their suitability for use as positive controls. The TMV SARS-CoV-2 PsVs require disruption of the coat to allow release of RNA, exactly as happens with the SARS-CoV-2 virions, and the inventors have shown that the same conditions used to analyse SARS-CoV-2 patient samples can successfully be used to amplify sequences contained within the TMV SARS-CoV-2 PsVs described herein. In addition, those of skill in the art will appreciate that the amplification of the target sequences contained within the TMV SARS-CoV-2 PsVs could further be used to calculate viral genome loads in patient samples, given that the concentrations of RNA from the target SARS-CoV-2 sequences would be known.

From several experiments, the inventors have shown herein that a single batch purification from 1 kg of plants would yield approximately 100 ml of purified TMV SARS-CoV-2 PsVs. As preliminary testing has shown that only 2×10⁻⁵ μl of undiluted sample is required per test reaction, a single batch purification from 1 kg of plants would be sufficient for approximately 5 million tests as a control. Furthermore, incorporation of the EGFP reporter gene into the RNA-expressing construct allows for the use of the TMV SARS-CoV-2 PsVs as an expression control for in vitro work in mammalian cells, with expression of EGFP from transfected TMV SARS-CoV-2 PsV constructs.

It will be appreciated that although the inventors of the present invention have demonstrated that TMV PsVs can serve as highly suitable and successful positive controls in SARS-CoV-2 RNA detection kits, the tobamovirus PsVs of the present invention are not limited to controls in the detection of SARS CoV-2 only. It is submitted that any synthetic DNA may be used to produce the mRNA contained within the PsVs using the same approach. Thus, the method of the present invention could be used to produce tobamovirus PsVs that could serve as positive controls in RNA kits used to detect other ssRNA viruses infecting humans, animals and even plants. For example, by incorporating appropriate sequences from human (or animal) influenza and parainfluenza viruses, respiratory syncytial virus, SARS-CoV-1, SARS-CoV-2 and other coronaviruses, rhinoviruses, and even adenoviruses, where mRNA sequences are targeted.

Furthermore, in one embodiment the method described herein may be used to develop stabilized RNA vaccines for animals and possibly even humans. mRNA of a viral protein for inducing neutralizing antibodies may be stably contained within tobamovirus PsVs and delivered to a subject via oral or parenteral vaccination. This would result in the translation of vaccine protein in the vaccinated host, thereby stimulating an immune response against the viral antigen and potentially providing protection against subsequent infection with the viral pathogen. By way of example, due to their stability, a tobamovirus PsV including SARS-CoV-2 antigen would present an advantage over the SARS-CoV-2 vaccines developed by Pfizer/BioNTech and Moderna which must be kept at −80° C. The encapsidated vaccine in contrast would be very stable at 4° C. for extended time making it a more viable option especially for resource-poor areas.

Moreover, TMV particles are known to efficiently be taken up by human and animal dendritic cells and can express payload genes within these cells. Specifically, TMV virus-like nanoparticles (VNPs) are efficiently taken up by dendritic cells (DCs) in vitro and are preferentially taken up by dendritic cells in draining lymph nodes in vivo in mice, where DC activation can result in potent antigen specific CD8+ T-cell responses if the VNPs display a foreign peptide-interestingly, of greater magnitude than adenovirus priming. The TMV CP may be genetically or chemically altered to contain sequences that enhance binding to specific cell types, including various tumour cells, which would enhance their use as therapeutic vaccines. For example, an integrin-binding motif displayed at the coat protein C-terminus allows more specific tumour cell targeting.

In the context of the present invention, TMV VNPs can be used as PsVs (VNPs encapsidating a foreign RNA). This is achieved by inclusion of a TMV “origin of assembly” or OriA sequence in any RNA target, which results in the efficient encapsidation of the target RNA by TMV coat protein (CP), in vitro or in vivo. The OriA sequence comprises three stable hairpin loops, of which only loop 1 is absolutely required for rapid and specific assembly initiation of helical nucleocapsid formation in the presence of a TMV CP suspension at a suitable concentration, that contains a high proportion of 34-subunit 2-layer disks of the CP.

The recombinant PsVs of the present invention may also be used for transfection of mammalian cells by standard methods, and once inside the mammalian cell, they would be co-translationally disassembled by ribosomes with concomitant expression of EGFP from the encapsidated RNA. This could serve as an infection control for S-pseudotyped or native SARS-CoV-2 virions and would allow both fluorescence and RT-PCR or LAMP detection of RNA.

As used herein, the term “pseudovirion” or “PsV” refers to a tobamovirus coat protein virus-like nanoparticle in which single stranded RNA of interest has been encapsidated. The pseudovirions of the invention contain non-native genetic material which is stabilised by encapsidation with the tobamovirus coat protein. The non-native genetic material may include any single stranded RNA, including mRNA encoding a reporter gene, a ssRNA target of a diagnostic assay for the detection of a single stranded RNA virus, mRNA encoding a therapeutic gene, an antigenic polypeptide, a hormone, an antibody or an enzyme and/or any other heterologous gene of interest.

In this specification “encapsidated” refers to the ssRNA being enclosed within a capsid or virus-like particle formed by the tobamovirus coat protein.

A “protein,” “peptide” or “polypeptide” is any chain of two or more amino acids, including naturally occurring or non-naturally occurring amino acids or amino acid analogues, irrespective of post-translational modification (e.g., glycosylation or phosphorylation). The term “protein” should be read to include “peptide” and “polypeptide” and vice versa.

The term “tobamovirus” as used herein refers to a genus of positive strand plant viruses in the family Virgaviridae, and includes without limitation: Bellpepper mottle virus (BPeMV), Brugmansia mild mottle virus, Cactus mild mottle virus (CMMoV), Clitoria yellow mottle virus, Cucumber fruit mottle mosaic virus, Cucumber green mottle mosaic virus (CGMMV), Cucumber mottle virus, Frangipani mosaic virus (FrMV), Hibiscus latent Fort Pierce virus (HLFPV), Hibiscus latent Singapore virus (HLSV), Kyuri green mottle mosaic virus, Maracuja mosaic virus (MarMV), Obuda pepper virus (ObPV), Odontoglossum ringspot virus (ORSV), Opuntia chlorotic ringspot virus, Paprika mild mottle virus, Passion fruit mosaic virus, Pepper mild mottle virus (PMMoV), Plumeria mosaic virus, Rattail cactus necrosis-associated virus (RCNaV), Rehmannia mosaic virus, Ribgrass mosaic virus (HRV), Sammons's Opuntia virus (SOV), Streptocarpus flower break virus, Sunn-hemp mosaic virus (SHMV), Tobacco latent virus, Tobacco mild green mosaic virus, Tobacco mosaic virus (TMV), Tomato brown rugose fruit virus (ToBRFV), Tomato mosaic virus (ToMV), Tomato mottle mosaic virus, Tropical soda apple mosaic virus, Turnip vein-clearing virus (TVCV), Ullucus mild mottle virus, Wasabi mottle virus (WMoV), Yellow tailflower mild mottle virus, Youcai mosaic virus (YoMV) aka oilseed rape mosaic virus (ORMV), and Zucchini green mottle mosaic virus.

It will be appreciated that a “reporter gene” may be selected from any nucleic acid encoding a polypeptide or protein whose transcription, translation and/or post-translation activity can be detected. Examples of reporter genes include, but are not limited to, genes for luciferase, secreted alkaline phosphatase, green fluorescent protein, beta-galactosidase, and the like.

The method of the invention includes the step of introducing polynucleotide(s) encoding a tobamovirus CP, an ssRNA of interest, and an OriA RNA sequence into a plant cell to obtain a recombinant TMV PsV. Preferably, the polynucleotides are contained on one or more vectors.

The term “recombinant” means that something has been recombined. When used with reference to a nucleic acid construct, the term refers to a molecule that comprises nucleic acid sequences that are joined together or produced by means of molecular biological techniques. Recombinant nucleic acid constructs may include a nucleotide sequence which is ligated to, or is manipulated to become ligated to, a nucleic acid sequence to which it is not ligated in nature, or to which it is ligated at a different location in nature. Accordingly, a recombinant nucleic acid construct indicates that the nucleic acid molecule has been manipulated using genetic engineering, i.e., by human intervention. Recombinant nucleic acid constructs may be introduced into a host cell by transformation and preferably by transformation with a vector.

The term “vector” refers to a means by which polynucleotides or gene sequences can be introduced into a cell. There are various types of vectors known in the art including plasmids, viruses, bacteriophages and cosmids. Generally, polynucleotides or gene sequences are introduced into a vector by means of recombinant DNA technology. The polynucleotides or gene sequences may be introduced into a vector by means of a cassette. The term “cassette” refers to a polynucleotide or gene sequence that is expressed from a vector, for example, the polynucleotide or gene sequences encoding the tobamovirus coat protein and/or the polynucleotide or gene sequences encoding the ssRNA of interest and an OriA RNA sequence. A cassette generally comprises a gene sequence inserted into a vector, which in some embodiments, provides regulatory sequences for expressing the polynucleotide or gene sequences. In other embodiments, the polynucleotide or gene sequence provides the regulatory sequences for its expression. In further embodiments, the vector provides some regulatory sequences, and the nucleotide or gene sequence provides other regulatory sequences. “Regulatory sequences” include but are not limited to promoters, transcription termination sequences, enhancers, splice acceptors, donor sequences, introns, ribosome binding sequences, poly(A) addition sequences, and/or origins of replication.

The expression of the tobamovirus CP and the transcription of the polynucleotide or gene sequences encoding the ssRNA of interest and an OriA RNA sequence in the plant cell results in the polypeptide self-assembling into virus-like particles in the cell. Specifically, the tobamovirus CP specifically recognises and interacts with the OriA RNA sequence and initiates assembly of the virus-like particles encapsidating the ssRNA of interest. Copies of the ssRNA of interest are specifically encapsidated directly into the virus-like particle during assembly in the cell to form pseudovirions. This is in contrast to indirect methods of incorporating a polynucleotide of interest into a pseudovirion by chemically or mechanically separating the virus-like particles and introducing a polynucleotide of interest into the virus-like particle to form a pseudovirion by a disassembly/reassembly method.

As used herein the terms “nucleic acid”, “nucleic acid molecule” or “polynucleotide” encompass both ribonucleotides (RNA) and deoxyribonucleotides (DNA), including cDNA, genomic DNA, and synthetic DNA. A nucleic acid may be double-stranded or single-stranded. Where the nucleic acid is single-stranded, the nucleic acid may be the sense strand or the antisense strand. A nucleic acid molecule may be any chain of two or more covalently bonded nucleotides, including naturally occurring or non-naturally occurring nucleotides, or nucleotide analogs or derivatives. The term “DNA” refers to a sequence of two or more covalently bonded, naturally occurring or modified deoxyribonucleotides.

In one embodiment, the tobamovirus PsVs as described herein may be used as a control in a diagnostic assay for the detection of a single stranded RNA virus, such as, but not limited to, SARS-CoV-2. The tobamovirus PsVs of this invention may also be used as positive controls for assays to detect other ssRNA viruses infecting humans, animals and/or plants, including other human respiratory viruses such as influenza viruses, respiratory syncytial virus, measles virus, human immunodeficiency virus (HIV), and other agents. Further, the tobamovirus PsVs may be used as a control for the detection of other RNA targets in a molecular diagnostic assay based on the detection using probes or amplification of single stranded RNA, such as the BRCA-ABL human ss-mRNA.

Particularly, the recombinant tobamovirus PsVs may be used for the calibration of diagnostic devices used to diagnose a single stranded RNA virus, such as SARS-CoV-2, in a subject and for active surveillance monitoring of single stranded RNA viral infections, including SARS-CoV-2 infections. For example, the recombinant tobamovirus PsVs may be used as a control in the GeneXpert® system, Thermo Fisher, Cobas SARS-CoV-2, Abbot Realtime SARS-CoV-2, BGI, CDC, SeeGene, Tib Mol Bio and Ultragene ABL assays. The pseudovirions of the present invention preferably include the same target nucleic acids as the assay system using the same complementary nucleic acid probes. Preferably, the tobamovirus PsV has the same diagnostic profile as the clinical sample containing the single stranded RNA, indistinguishably, and with the same specificity in an assay, when using the same detection probes or primers for both the clinical sample and the tobamovirus PsV. In one embodiment, the target nucleic acids may include any target nucleic acids in the SARS-CoV-2 genome, including but not limited to a nucleic acid target within a gene selected from: RNA-dependent RNA polymerase (RdRp), nucleocapsid (N) gene, envelope (E) gene, and spike (S) gene.

As used herein the term “single stranded RNA diagnostic target” refers to a target region of single stranded RNA that is detected by a molecular diagnostic assay, in particular a nucleic acid assay that directly detects the presence of the RNA using either a probe or primer that specifically binds the region of single stranded RNA. Preferably the assay is an RT-PCR assay.

The term “complementary” refers to two nucleic acids molecules, e.g., DNA or RNA, which are capable of forming Watson-Crick base pairs to produce a region of double-strandedness between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

As used herein a “substantially identical” sequence is an amino acid or nucleotide sequence that differs from a reference sequence only by one or more conservative substitutions, or by one or more non-conservative substitutions, deletions, or insertions located at positions of the sequence that do not destroy or substantially reduce the binding of one or more of the probes for the targets in the molecular diagnostic assay. Alignment for purposes of determining percent sequence identity can be achieved in various ways that are within the knowledge of those with skill in the art. These include using, for instance, computer software such as ALIGN, Megalign (DNASTAR), CLUSTALW or BLAST software. Those skilled in the art can readily determine appropriate parameters for measuring alignment, including any algorithms needed to achieve maximal alignment over the full length of the sequences being compared. In one embodiment of the invention there is provided for a polynucleotide sequence that has at least about 80% sequence identity, at least about 90% sequence identity, or even greater sequence identity, such as about 95%, about 96%, about 97%, about 98% or about 99% sequence identity to the sequences described herein.

Alternatively, or additionally, two nucleic acid sequences may be “substantially identical” if they hybridize under high stringency conditions. The “stringency” of a hybridisation reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridisation generally depends on the ability of denatured DNA to re-anneal when complementary strands are present in an environment below their melting temperature. A typical example of such “stringent” hybridisation conditions would be hybridisation carried out for 18 hours at 65° C. with gentle shaking, a first wash for 12 min at 65° C. in Wash Buffer A (0.5% SDS; 2×SSC), and a second wash for 10 min at 65° C. in Wash Buffer B (0.1% SDS; 0.5% SSC).

The invention also relates in part to a method of eliciting an immune response in a subject comprising administering to a subject in need thereof an immunologically effective amount or a prophylactically effective amount of the PsVs of the present invention or compositions containing them.

The tobamovirus PsVs or compositions of the invention can be provided either alone or in combination with other compounds (for example, nucleic acid molecules, small molecules, peptides, or peptide analogues), in the presence of an adjuvant, or any carrier, such as a pharmaceutically acceptable carrier and in a form suitable for administration to mammals, for example, humans, cattle, sheep, etc.

As used herein a “pharmaceutically acceptable carrier” or “excipient” includes any and all antibacterial and antifungal agents, coatings, dispersion media, solvents, isotonic and absorption delaying agents, and the like that are physiologically compatible. A “pharmaceutically acceptable carrier” may include a solid or liquid filler, diluent or encapsulating substance which may be safely used for the administration of the tobamovirus PsVs or vaccine compositions to a subject. The pharmaceutically acceptable carrier can be suitable for intramuscular, intraperitoneal, intravenous, subcutaneous, oral or sublingual administration. Pharmaceutically acceptable carriers include sterile aqueous solutions, dispersions and sterile powders for the preparation of sterile solutions. The use of media and agents for the preparation of pharmaceutically active substances is well known in the art. Where any conventional media or agent is incompatible with the active compound, use thereof in the pharmaceutical compositions of the invention is not contemplated. Supplementary active compounds can also be incorporated into the compositions.

Suitable formulations or compositions to administer the tobamovirus PsVs and vaccine compositions to subjects fall within the scope of the invention. Any appropriate route of administration may be employed, such as, parenteral, intravenous, subcutaneous, intramuscular, intracranial, intraorbital, ophthalmic, intraventricular, intracapsular, intraspinal, intrathecal, intracistemal, intraperitoneal, intranasal, aerosol, topical, or oral administration.

As used herein the term “subject” includes mammals, such as humans, cattle, sheep, etc. Preferably the subject is a human subject.

For vaccine formulations, an effective amount of the tobamovirus PsVs or compositions of the invention can be provided, either alone or in combination with other compounds, with immunological adjuvants, for example, aluminium hydroxide dimethyldioctadecylammonium hydroxide or Freund's incomplete adjuvant. The tobamovirus PsVs or compositions of the invention may also be linked with suitable carriers and/or other molecules, in order to enhance immunogenicity.

By “immunogenically effective amount” is meant an amount effective, at dosages and for periods of time necessary, to achieve a desired immune response. The desired immune response may include stimulation or elicitation of an immune response, for instance a T or B cell response.

A “prophylactically effective amount” refers to an amount effective, at dosages and for periods of time necessary, to achieve a desired prophylactic result, such as prevention of onset of a condition associated with an infection.

The amount of active compound in the composition may vary according to factors such as the disease state, age, sex, and weight of the individual. Dosage regimens may be adjusted to provide the optimum immunogenic or prophylactic response. For example, a single dose may be administered, or multiple doses may be administered over time. It may be advantageous to formulate the compositions in dosage unit forms for ease of administration and uniformity of dosage.

The term “preventing”, when used in relation to an infectious disease, or other medical disease or condition, is well understood in the art, and includes administration of a composition which reduces the frequency of or delays the onset of symptoms of a condition in a subject relative to a subject which does not receive the composition. Prevention of a disease includes, for example, reducing the number of diagnoses of the infection in a treated population versus an untreated control population, and/or delaying the onset of symptoms of the infection in a treated population versus an untreated control population.

The term “prophylactic” treatment is well known to those of skill in the art and includes administration to a subject of one or more of the compositions of the invention. If the composition is administered prior to clinical manifestation of the unwanted condition (e.g., disease or other unwanted state of the subject) then the treatment is prophylactic, i.e., it protects the host against developing the unwanted condition.

Toxicity and immunogenic efficacy of compositions of the invention may be determined by standard pharmaceutical procedures in cell culture or using experimental animals, such as by determining the LD50 and the ED50. Data obtained from the cell cultures and/or animal studies may be used to formulating a dosage range for use in a subject. The dosage of any composition of the invention lies preferably within a range of circulating concentrations that include the ED50, but which has little or no toxicity. The dosage may vary within this range depending upon the dosage form employed and the route of administration utilized.

The following examples are offered by way of illustration and not by way of limitation.

### Example 1

### Design and Synthesis of SARS-CoV-2 Artificial DNA Sequence

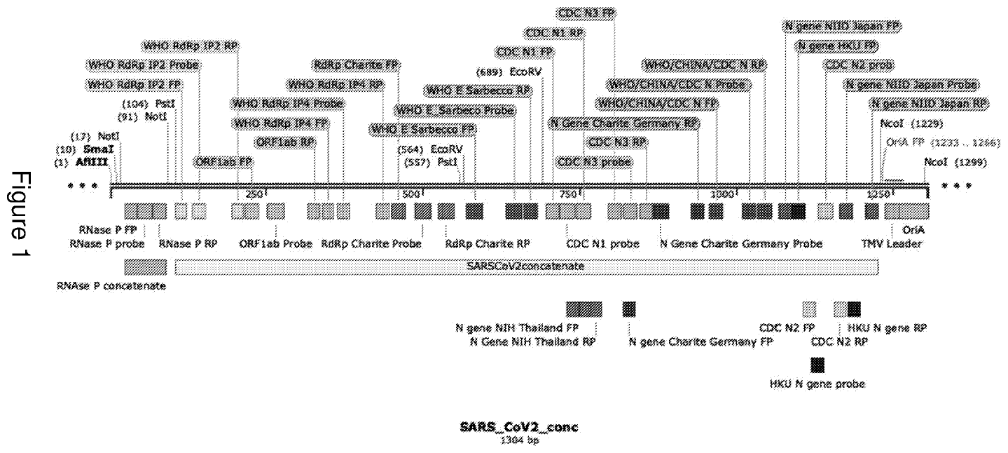

An artificial DNA sequence containing a concatenate of 13 SARS-CoV-2-specific primer/probe sequence sets that represent some of the most commonly used targets for amplification in SARS-CoV-2 RT-qPCR tests reported in scientific literature, was designed and synthesized by GenScript (SEQ ID NO:1). The construct also contained the RNAse P internal control primer/probe sequence set and was designed to have some flexibility by the incorporation of various restriction enzyme sites whereby certain of these primer/probe sequence sets could be removed (FIG. 1).

### Example 2

### Cloning and Expression of TMV PsVs

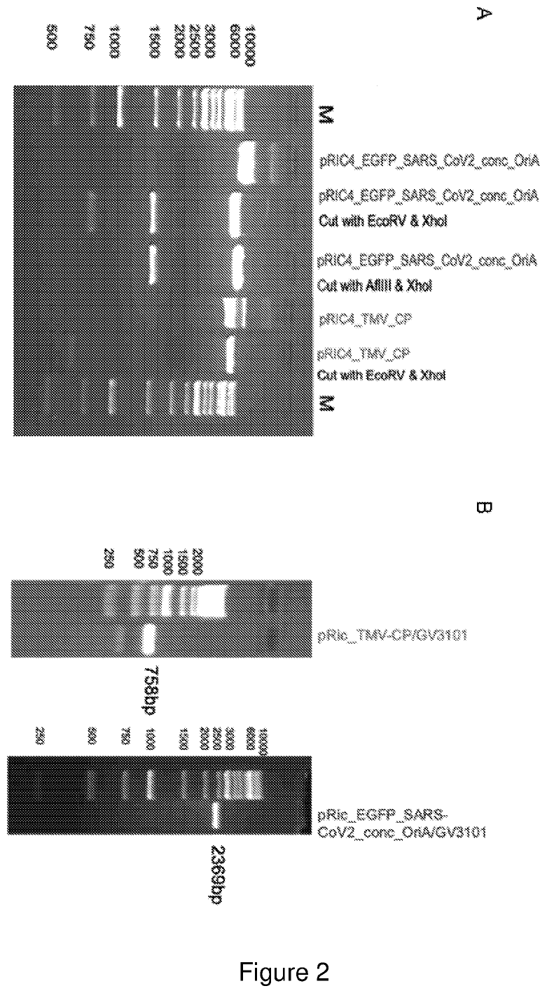

The artificial DNA sequence (SEQ ID NO:1) was cloned into the pRIC4 plant transient expression vector downstream from an EGFP reporter gene (SEQ ID NO:2) and upstream from the TMV OriA sequence (SEQ ID NO:3). A DNA version of the native TMV CP gene (GenBank Accession number: V01408.1) (SEQ ID NO:4) was also cloned into the pRIC4 vector and both constructs were electroporated into Agrobacterium strain GV3101::pMP90RK (FIG. 2). The PRIC4 vector is a modified vector based on the pRIC3 vector previously described in Regnard et al (2010), which is incorporated herein in its entirety by reference. The pRIC4 vector was obtained by inserting a 1455 bp sequence (SEQ ID NO:10) containing the Tomato Aspermy virus (TAV) 2b gene (NCBI accession number AJ320274.1), which is an RNA silencing suppressor, between nucleotide positions 2878 and 4334 of the pRIC3 vector. The full sequence of the pRIC4 vector is provided as SEQ ID NO:11.

The two recombinant Agrobacterium strains including the pRIC4_TMV_CP and the pRIC4_EGFP_SARS-CoV2_conc_OriA, respectively, were used to co-infiltrate the leaves of five-week-old Nicotiana benthamiana plants at different OD₆₀₀ combinations, as described previously in Dennis et al. 2019 At 3 days' post-infiltration, leaves were harvested and homogenized together in two volumes of PBS buffer pH 7.4 containing 1× Complete Mini, EDTA-free protease inhibitor cocktail (Roche) and incubated at 4° C. for 1 h with gentle agitation. Crude plant extracts were clarified by centrifugation at 10 000 rpm for 30 min at 4° C. in a JA14 rotor (Beckman) and the resulting supernatant was then filtered through a layer of Miracloth™ (Merck, Germany) before being clarified further by a second centrifugation step at 13000 rpm for 20 min at 4° C.

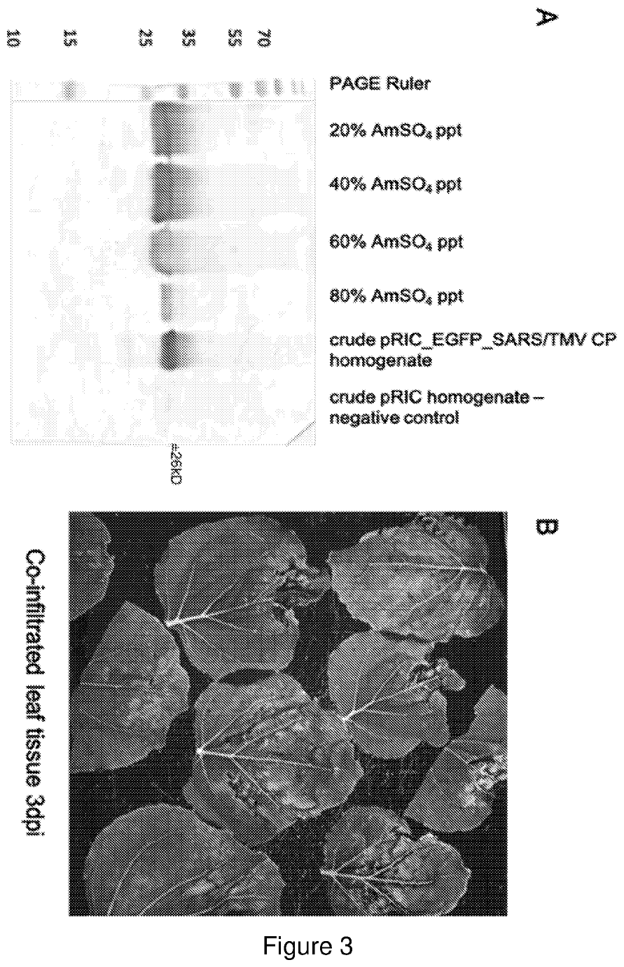

To demonstrate the ability of the encapsidated RNA to be translated into a protein the inventors showed that eGFP was expressed from the artificial gene cloned into the plant expression vector pRIC4. A sample from each clarified lysate was removed and ammonium sulphate (NH₄)₂SO₄ precipitation (20-80% in 20% incrementing steps) was used to demonstrate EGFP expression. The amount of solid (NH₄)₂SO₄ required was calculated using the online ammonium sulfate calculator tool by EnCor Biotechnology Inc. In each case, protein was precipitated at 4° C. for 30 min followed by centrifugation at 13000 rpm for 20 min at 4° C. in a JA14 rotor (Beckman). Protein pellets were resuspended in ½ volume PBS and analysed by western blot using a commercial mouse anti-EGFP antiserum diluted (1:10000) in blocking buffer together with a commercial anti-mouse alkaline phosphatase-conjugated secondary antiserum again diluted (1:10000) in blocking buffer. The results are depicted in FIG. 3, which indicates that EGFP was best purified using 20-40% (NH₄)₂SO₄. The SARS-CoV-2 artificial DNA sequence is provided as SEQ ID NO:1. The sequences of the EGFP reporter gene (SEQ ID NO:2), TMV OriA sequence (SEQ ID NO:3) and DNA version of the native TMV CP gene (SEQ ID NO:4) are provided in Table 1.

TABLE 1
Sequences of the EGFP reporter gene, TMV OriA sequence
(including 24 nucleotide leader sequence and 38 nucleotide
following sequence on either side of the 126 nucleotide
OriA sequence) and DNA version of the native TMV CP gene.

| Description | Sequence (5′-3′) |
|---|---|
| EGFP reporter | ATGGTGAGCAAGGGTGAGGAGCTGTTCACCGGGGT |
| gene | GGTGCCCATCCTGGTCGAGCTGGACGGCGACGTTA |
| (SEQ ID NO: 2) | ACGGCCACAAGTTTAGCGTGTCCGGCGAGGGCGA |
| | GGGCGATGCCACCTACGGCAAGCTGACCCTGAAGT |
| | TCATCTGCACCACCGGCAAGCTGCCCGTGCCCTGG |
| | CCCACCCTCGTGACCACCCTGACCTACGGCGTGCA |
| | GTGCTTCAGCCGCTACCCCGACCACATGAAGCAGC |
| | ACGACTTCTTCAAGTCCGCCATGCCCGAAGGCTAC |
| | GTCCAGGAGCGCACCATCTTCTTCAAGGACGACGG |
| | CAACTACAAGACCCGCGCCGAGGTGAAGTTCGAGG |
| | GCGACACCCTGGTGAACCGCATCGAGCTGAAGGGC |
| | ATCGACTTCAAGGAGGACGGCAACATCCTGGGGCA |
| | CAAGCTGGAGTACAACTACAACAGCCACAACGTCTA |
| | TATCATGGCCGACAAGCAGAAGAACGGCATCAAGG |
| | TGAACTTCAAGATCCGCCACAACATCGAGGACGGC |
| | AGCGTGCAGCTCGCCGACCACTACCAGCAGAACAC |
| | CCCCATCGGCGACGGCCCCGTGCTGCTGCCCGAC |
| | AACCACTACCTGAGCACCCAGTCCGCCCTGAGCAA |
| | AGACCCCAACGAGAAGCGCGATCACATGGTCCTGC |
| | TGGAGTTCGTGACCGCCGCCGGGATCACTCTCGGC |
| | ATGGACGAGCTGTACAAGTAA |
| TMV OriA | TTATAGAAATAATATAAAATTAGGTTTGAGAGAGAAG |
| sequence | ATTACAAACGTGAGAGACGGAGGGCCCATGGAACT |
| (SEQ ID NO: 3) | TACAGAAGAAGTCGTTGATGAGTTCATGGAAGATGT |
| | CCCTATGTCGATCAGGCTTGCAAAGTTTCGATCTCG |
| | AACCGGAAAAAAGAGTGATGTCCGCAAAGGGAAAA |
| | ATAGTAGTA |
| DNA version of | ATGAGTTACTCTATTACAACTCCGAGTCAATTTGTTT |
| the native TMV | TCCTTAGTTCTGCTTGGGCTGATCCGATTGAACTTA |
| CP gene | TTAATCTATGCACAAACGCTCTAGGTAATCAATTTCA |
| (SEQ ID NO: 4) | AACACAACAAGCTCGCACTGTTGTACAAAGACAATT |
| | CTCTGAGGTTTGGAAACCGAGTCCGCAAGTTACAGT |
| | AAGATTTCCGGATTCTGATTTCAAGGTATACCGTTA |
| | CAACGCTGTTCTTGATCCGCTAGTAACAGCTCTTCT |
| | AGGAGCTTTCGATACTAGAAACCGTATTATTGAAGT |
| | AGAGAACCAAGCTAACCCGACAACTGCTGAAACACT |
| | TGATGCTACTCGTCGCGTTGATGATGCTACAGTAGC |
| | TATTCGCTCTGCTATTAATAACCTTATTGTTGAACTA |
| | ATTCGTGGTACTGGAAGTTACAACCGCAGTTCTTTC |
| | GAGAGTTCTAGTGGTCTAGTATGGACAAGTGGACC |
| | GGCTACT |

### Example 3

### Purification and Characterization of TMV SARS-CoV2 PsVs

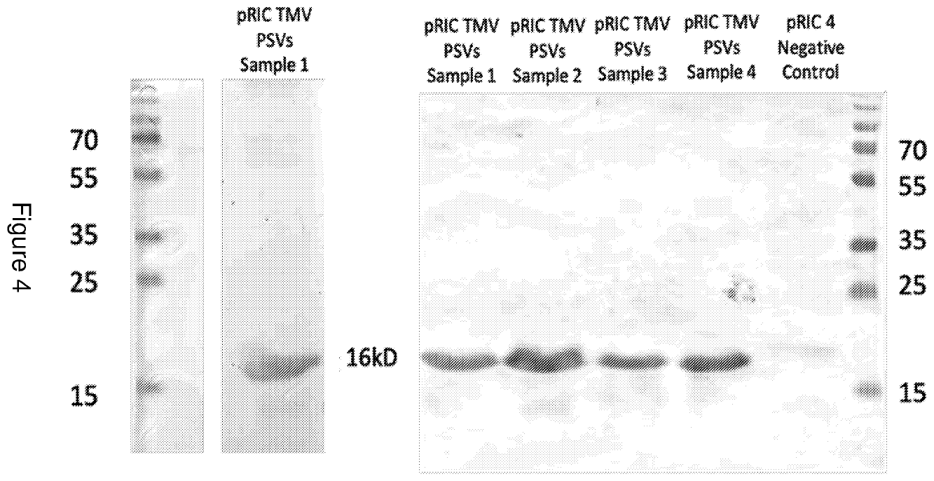

TMV SARS-CoV2 PsVs were purified from the remaining crude plant lysates by the addition of 20% Polyethylene glycol (PEG) MW 8000 and 2.5 M NaCl to final concentrations of 4% (w/v) PEG and 0.5M NaCl and incubation overnight at 4° C. with gentle agitation. The PsVs were pelleted by centrifugation at 4° C. for 10 min at 10 000 rpm in a JA14 rotor (Beckman) and resuspended in 1/10th volume PBS containing protease inhibitor. PsVs were precipitated a second time with 4% PEG/0.5M NaCl overnight at 4° C. with gentle agitation followed by centrifugation at 10 000 rpm for 10 min and 4° C. and resuspended in ½ volume PBS containing protease inhibitor. PsVs were precipitated a third time with 4% PEG/0.5M NaCl overnight at 4° C. with gentle agitation followed by centrifugation at 10 000 rpm for 10 min and 4° C. The PsV pellet was resuspended in ⅔ volume PBS and 50 μL samples were analysed by SDS-PAGE, followed by Coomassie blue staining. Samples were also analysed by western blot using TMV rabbit antiserum diluted (1:10000) in blocking buffer together with a commercial anti-rabbit alkaline phosphatase-conjugated secondary antiserum again diluted (1:10000) in blocking buffer (FIG. 4).

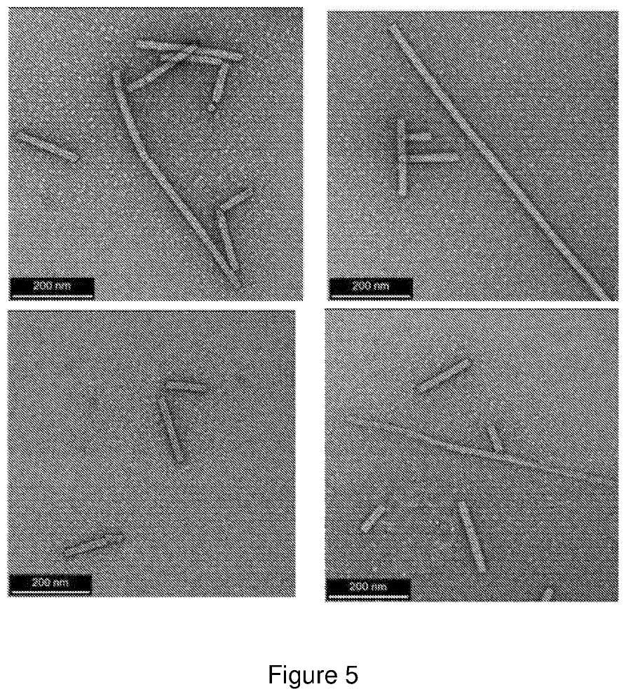

TMV SARS-CoV2 PsVs were also imaged by Transmission Electron Microscopy (TEM) (FIG. 5).

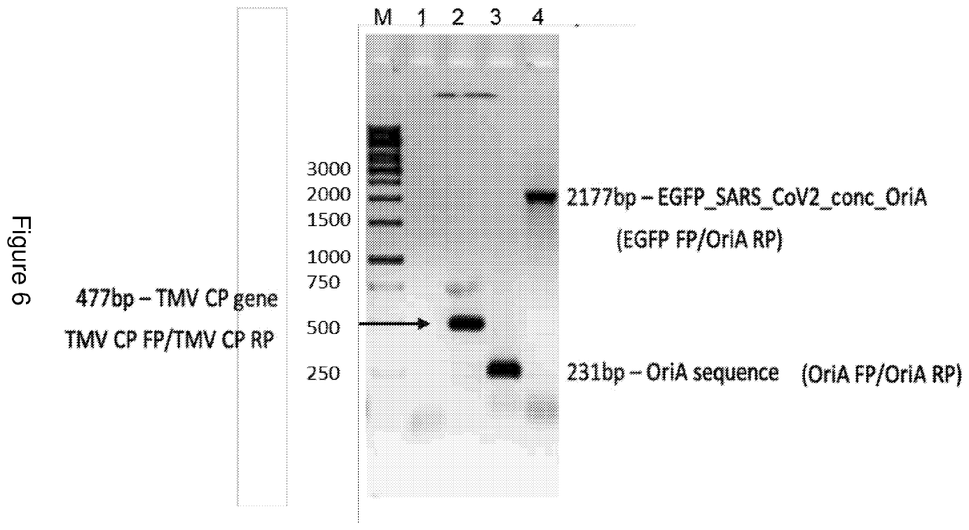

To demonstrate the presence of EGFP_SARS_CoV2_conc_OriA mRNA within the PsVs, cDNA was synthesized by reverse transcription of putative ssRNA contained within the PsVs, followed by PCR amplification. The OriA reverse primer (SEQ ID NO:8) was used to synthesise cDNA from pRIC4 TMV SARS-CoV2 PsVs, while the TMV CP reverse primer (SEQ ID NO:6) was used to synthesize cDNA from TMV CP PsVs containing TMV CP/OriA mRNA previously produced (positive control). As a negative control, water was used instead of PsVs. Thereafter, the TMV COP forward/reverse primer set (SEQ ID NO:5 and SEQ ID NO:6), OriA forward/reverse primer set (SEQ ID NO:7 and SEQ ID NO:8), or EGFP forward/OriA reverse primer set (SEQ ID NO:9 and SEQ ID NO:8) was used for PCR amplification of the transcribed cDNA and yielded 477 bp (TMV OP), 231 bp (OriA) or 2177 bp (EGFP_SARS_CoV2_conc_OriA) amplification products respectively as depicted in FIG. 6. The primer sets used for reverse transcription and PCR amplification are provided in Table 2 below.

TABLE 2
Primers used for reverse transcription
and PCR amplification of EGFP
SARS_CoV2_conc_OriA mRNA within the PsVs

| Primer | Sequence (in the 5′-3′ direction) | SEQ ID NO |
|---|---|---|
| TMV CP | ATGAGTTACTCTATTACAACTCCGAGTCAAT | SEQ ID |
| forward | TTG | NO: 5 |
| primer | | |
| TMV CP | AGTAGCCGGTCCACTTGTCCA | SEQ ID |
| reverse | | NO: 6 |
| primer | | |
| OriA | CAAGTAAACATGTCCCGGGGAATTATAGAA | SEQ ID |
| forward | ATAATATAAAATTAGGTTTGAG | NO: 7 |
| primer | | |
| OriA | CTCTAGAGAGCTCGACTCGAGTACTACTAT | SEQ ID |
| reverse | TTTTCCCTTTGCG | NO: 8 |
| primer | | |
| EGFP | AAAAAAAAAAACATGGTGAGCAAGGGTGAG | SEQ ID |
| forward | GAG | NO: 9 |
| primer | | |

### Example 4

### Testing of TMV SARS-Co V2 PsVs in SARS-Co V2 Detection Assays

Preliminary testing of the TMV SARS-CoV2 PsVs was conducted at the National Health Laboratory Services facility in Cape Town and Johannesburg in best-practice routine SARS-CoV2 detection assays. The following assays were used to test the TMV SARS-CoV2 PsVs: Allplex™ 2019-nCov Assay (Seegene Inc), Xpert® Xpress SARS-CoV-2 for GeneXpert® system (Cepheid), BioGX SARS-CoV-2 Reagents for BD MAX™ System (Becton, Dickinson and Company), TaqPath Covid19 CE IVD RT-PCR kit (Thermo Fisher). Additional testing is underway using Cobas® SARS-CoV-2 for Cobas system (Roche) and Abbot RealTime SARS-CoV-2 (Abbot).

Real Time qPCR of TMV SARS-CoV2 PsV samples successfully amplified the targets tested. Routine extraction methods were used whereby 50 μl TMV SARS-CoV2 PsV sample aliquots were incubated at 98° C. for 5 min followed by 4° C. for 2 minutes before adding a 2 μl sample to the 28 μl reaction, that is, only 2 μl samples were required per test. Samples were tested as undiluted, 10⁻¹, 10⁻², 10⁻³, 10⁻⁴, 10⁻⁵ and 10⁻⁶ dilutions. All samples appeared similar in concentration, demonstrating similar Ct values with samples that were tested at 10⁻⁵ dilutions demonstrating Ct values closest to what is observed for typical diagnostic samples. It would therefore appear that only 2×10⁻⁵ μl of undiluted TMV SARS-CoV2 PsV sample is required per reaction, indicating the suitability of the TMV SARS-CoV2 PsVs described in this invention for use as positive controls in SARS-CoV-2 RNA detection kits.

### REFERENCES

Dennis, S. J. (2019). Development of plant-produced African horse sickness virus vaccines. Doctor of Philosophy, University of Cape Town.
Regnard, G. L., Halley-Stott, R. P., Tanzer, F. L., Hitzeroth, I. I., and Rybicki, E. P. (2010). High level protein expression in plants through the use of a novel autonomously replicating geminivirus shuttle vector. Plant Biotechnology Journal, 8, pp. 38-46.

1. A method for stabilising a single stranded RNA (ssRNA) by encapsidation of the ssRNA with a tobamovirus coat protein to obtain a pseudovirion (PsV), the method comprising the steps of:
i) providing:
a) a first nucleic acid encoding a tobamovirus coat protein; and
b) a second nucleic acid encoding a single stranded RNA (ssRNA) comprising a tobamovirus encapsidation origin (OriA),
wherein the first and second nucleic acids are contained on at least one expression vector;
ii) expressing the tobamovirus coat protein; and
iii) co-expressing the ssRNA,
wherein the expressed tobamovirus coat protein interacts with the OriA sequence on the ssRNA to initiate encapsidation of the ssRNA by the tobamovirus coat protein, thereby forming a pseudovirion.

2. The method of claim 1, wherein the first and second nucleic acids are operably linked to regulatory sequences that allow for expression of the tobamovirus coat protein and the ssRNA, respectively.

3. The method of claim 1, wherein the ssRNA is one or more ssRNA selected from the group consisting of: a mRNA encoding a reporter gene, a ssRNA target of a molecular diagnostic assay for the detection of a ssRNA virus, a mRNA target of a diagnostic assay for the detection of a disease or disorder, mRNA encoding a therapeutic gene, mRNA encoding an antigenic polypeptide, mRNA encoding a hormone, mRNA encoding an antibody and mRNA encoding an enzyme.

4. The method of claim 3, wherein the reporter gene is selected from an enhanced green fluorescent protein gene, luciferase gene, a secreted alkaline phosphatase gene, a gene encoding a fluorescent protein or a horseradish peroxidase gene.

5. The method of claim 3, wherein the ssRNA target of the molecular diagnostic assay for the detection of a ssRNA virus is a ssRNA virus sequence detected by the molecular diagnostic assay.

6. The method of claim 5, wherein the ssRNA virus sequence is one or more sequence of a virus selected from the group consisting of an influenza virus, a parainfluenza virus, a respiratory syncytial virus, a measles virus, a coronavirus, a rhinovirus, an adenovirus, HCV, HIV, and Ebola virus.

7. The method of claim 6, wherein the ssRNA virus sequence is a SARS-CoV-2 sequence of SEQ ID NO:1.

8. The method of claim 1, wherein the tobamovirus coat protein and the ssRNA are expressed in a plant cell and the method comprises recovering the PsVs from the plant cell.

9. The method of claim 1, wherein the tobamovirus is Tobacco mosaic virus (TMV).

10. A pseudovirion produced according to the method of claim 1, wherein the pseudovirion comprises a tobamovirus coat protein capsid which encapsidates the ssRNA comprising the OriA.

11. A diagnostic control composition comprising a pseudovirion, wherein the pseudovirion comprises a tobamovirus coat protein capsid which encapsidates a single stranded RNA (ssRNA) comprising a tobamovirus encapsidation origin (OriA), wherein the ssRNA is a sequence detected by a molecular diagnostic assay and wherein the diagnostic control composition is a positive control for the molecular diagnostic assay.

12. The diagnostic control composition of claim 11, wherein the ssRNA is one or more ssRNA selected from the group consisting of a mRNA encoding a reporter gene, a ssRNA target of a molecular diagnostic assay for the detection of a ssRNA virus, and a mRNA target of a diagnostic assay for the detection of a disease or disorder.

13. The diagnostic control composition of claim 11, wherein the reporter gene is selected from an enhanced green fluorescent protein gene, luciferase gene, a secreted alkaline phosphatase gene, a gene encoding a fluorescent protein or a horseradish peroxidase gene.

14. The diagnostic control composition of claim 12, wherein the ssRNA target of the molecular diagnostic assay for the detection of a ssRNA virus is an ssRNA virus sequence detected by the molecular diagnostic assay.

15. The diagnostic control composition of claim 14, wherein the ssRNA virus sequence is one or more sequence of a virus selected from the group consisting of an influenza virus, a parainfluenza virus, a respiratory syncytial virus, a measles virus, a coronavirus, a rhinovirus, an adenovirus, HCV, HIV, and Ebola virus.

16. The diagnostic control composition of claim 15, wherein the ssRNA virus sequence is a SARS-CoV-2 sequence of SEQ ID NO:1.

17. The diagnostic control composition of claim 11, wherein the tobamovirus is Tobacco mosaic virus (TMV).

18. A pharmaceutical composition comprising a pseudovirion of claim 10, and a pharmaceutically acceptable carrier or adjuvant.

19. A method of eliciting an immune response in a subject, the method comprising administering an immunologically effective amount of a pseudovirion of claim 10 to the subject.


SEQUENCE LISTING (11 sequences)

SEQ ID NO: 1 - DNA, 1234 bp, Artificial sequence
  Artificial DNA sequence containing SARS-CoV-2 target sequences
acatgttcccgggtagcggccgcgagatttggacctgcgagcgggttctgacctgaaggctctgcgcgggagcggctgtctccacaagtgcggccgctactgcagatgagcttagtcctgttgcactacgacagatgtcttgtgctgccggtactacacaaactgcttgcactgatgacaatgcgttagcttactacaacacaacaaagggagtaaccctgtgggttttacacttaaaaacacagtctgtaccgtctgcggtatgtggaaaggttatggctgtagttgtgatcaactccgcgaacccatgcttcagtcagctgatgcacaatcgttgaggtaactggtatgatttcggtgatttcatacaaaccacgccaggtagtggagttcctgttgtagattcttattattcattgttaatgcctatattaaccttgaccagatgagtgaaatggtcatgtgtggcggttcactatatgttaaaccaggtggaacctcatcaggagatgccacaactgcttatgctaatagtgtttttaacatttggagctgcagggagatatcagacaggtacgttaatagttaatagcgtacttctttttcttgctttcgtggtattcttgctagttacactagccatccttactgcgcttcgattgtgtgcgtactgctgcaatattgttgatatccaatgaccccaaaatcagcgaaatcaccccgcattacgtttggtggaccctcagattcaactggcagtaaccagaatggagaacgcagtggggcgtaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcattagagtaggggaacttctcctgctagaatggctggcaatggcggtgatgctgctcttgctttgctgctgcttgacagattgaaccagcttgagagcaaaatgtctggtaaagccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccaggaccatgg

SEQ ID NO: 2 - DNA, 720 bp, Artificial sequence
  EGFP reporter gene
atggtgagcaagggtgaggagctgttcaccggggtggtgcccatcctggtcgagctggacggcgacgttaacggccacaagtttagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 3 - DNA, 188 bp, Tobacco Mosaic Virus
ttatagaaataatataaaattaggtttgagagagaagattacaaacgtgagagacggagggcccatggaacttacagaagaagtcgttgatgagttcatggaagatgtccctatgtcgatcaggcttgcaaagtttcgatctcgaaccggaaaaaagagtgatgtccgcaaagggaaaaatagtagta

SEQ ID NO: 4 - DNA, 477 bp, Artificial sequence
  DNA version of TMV coat protein (CP) gene
atgagttactctattacaactccgagtcaatttgttttccttagttctgcttgggctgatccgattgaacttattaatctatgcacaaacgctctaggtaatcaatttcaaacacaacaagctcgcactgttgtacaaagacaattctctgaggtttggaaaccgagtccgcaagttacagtaagatttccggattctgatttcaaggtataccgttacaacgctgttcttgatccgctagtaacagctcttctaggagctttcgatactagaaaccgtattattgaagtagagaaccaagctaacccgacaactgctgaaacacttgatgctactcgtcgcgttgatgatgctacagtagctattcgctctgctattaataaccttattgttgaactaattcgtggtactggaagttacaaccgcagttctttcgagagttctagtggtctagtatggacaagtggaccggctact

SEQ ID NO: 5 - DNA, 34 bp, Artificial sequence
  TMV CP forward primer
atgagttactctattacaactccgagtcaatttg

SEQ ID NO: 6 - DNA, 21 bp, Artificial sequence
  TMV CP reverse primer
agtagccggtccacttgtcca

SEQ ID NO: 7 - DNA, 52 bp, Artificial sequence
  OriA forward primer
caagtaaacatgtcccggggaattatagaaataatataaaattaggtttgag

SEQ ID NO: 8 - DNA, 43 bp, Artificial sequence
  OriA reverse primer
ctctagagagctcgactcgagtactactatttttccctttgcg

SEQ ID NO: 9 - DNA, 33 bp, Artificial sequence
  EGFP forward primer
aaaaaaaaaaacatggtgagcaagggtgaggag

SEQ ID NO: 10 - DNA, 288 bp, Tomato Aspermy Virus
atggcaagcatcgagatccctctacacgagatcattcgaaagttggaacggatgaatcaaaagaaacaagcacagaggaaacgacacaaactgaaccgcaaggagcggggtcacaaaagtccaagtgaacaaaggcgatcggagttatggcacgcgcgtcaagttgaactttctgccattaattccgataattcttcagatgagggtaccactctgtgtcgctttgacacatttggttccaagtctgatgctatttgtgatcgctctgactggtgtctcgatcaatga

SEQ ID NO: 11 - DNA, 7392 bp, Artificial sequence
  pRIC4 vector
gaagagcgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgctcgacctggtccaaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgagttcacaacacaaatcagatttatagagagatttataaaaaaaaaaaaacatgtgatcccgggaattcgctagcactagtaagcttgtcgactcgagctctctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgaccgggcggcgggtcgagaatgattattttatgaatatatttcattgtgcaagtagatagaaattacatatgttacataacacacgaaataaacaaaaaaagacaatccaaaaacaaacaccccaaaaaaaataatcactttagataaactcgtatgaggagaggcacgttcagtgactcgacgattcccgagcaaaaaaagtctccccgtcacacatatagtgggtgacgcaattatctttaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaaatcgatggtaccgttccaatagttgtgtcgtccgagacttctagcccaggtggtctttccggtacgagttggtccgcagatgtagaggctggggtgtcggattccattccttccattgtcctggttaaatcggccatccattcaaggtcagattgagcttgttggtatgagacaggatgtatgtaagtataagcgtctatgcttacatggtatagatgggtttccctccaggagtgtagatcttcgtggcagcgaagatctgattctgtgaagggcgacacatacggttcaggttgtggagggaataatttgttggctgaatattccagccattgaagttttgttgcccattcatgagggaattcttccttgatcatgtcaagatattcctccttagacgttgcagtctggataatagttctccatcgtgcgtcagatttgcgaggagagaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggagaagagcatgatagtgggtagtgccatcttgatgaagctcagaagcaacaccaaggaagaaaataagaaaaggtgtgagtttctcccagagaaactggaataaatcatctctttgagatgagcacttgggataggtaaggaaaacatatttagattggagtctgaagttcttactagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactatacaagcatatacgatgtgatggtattgactagagtttaaacgaattcgagctcggtacccccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcttcgactctagaggatccccttaaatcgatatggcaagcatcgagatccctctacacgagatcattcgaaagttggaacggatgaatcaaaagaaacaagcacagaggaaacgacacaaactgaaccgcaaggagcggggtcacaaaagtccaagtgaacaaaggcgatcggagttatggcacgcgcgtcaagttgaactttctgccattaattccgataattcttcagatgagggtaccactctgtgtcgctttgacacatttggttccaagtctgatgctatttgtgatcgctctgactggtgtctcgatcaatgacaattgctcgaattcgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatgtttaaactatcagtgtttgacaggatatattggcgggtaaacctaagagaaaagagcgtttattagaataatcggatatttaaaagggcgtgaaaaggtttatccgttcgtccatttgtatgtgtacatcaccgacgagcaaggcaagaccgagcgcctttccgacgctcaccgggctggttgccctcgccgctgggctggcggccgtctatggccctgcaaacgcgccagaaacgccgtcgaagccgtgtgcgagacaccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgaaagctacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggactctagctagaggatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgatcgaatactaacgtctctaccacatt